(19) United States
(12) Patent Application Publication
(10) Pub. No.: US 2026/0060550 A1
(43) Pub. Date: Mar. 5, 2026

(54) BIOMETRIC SYSTEM

(71) Applicants: Reza Derakhshani, Shawnee, KS (US); Mostafizur Rahman, Leawood, KS (US); Keerti Bajaj, Kansas City, MO (US); Satya Sai Siva Rama Krishna Akula, Kansas City, MO (US); Sumanth Dasari, Kansas City, MO (US); Bhuvan Chennoju, Kansas City, MO (US)

(72) Inventors: Reza Derakhshani, Shawnee, KS (US); Mostafizur Rahman, Leawood, KS (US); Keerti Bajaj, Kansas City, MO (US); Satya Sai Siva Rama Krishna Akula, Kansas City, MO (US); Sumanth Dasari, Kansas City, MO (US); Bhuvan Chennoju, Kansas City, MO (US)

(73) Assignee: The Curators of the University of Missouri, Columbia, MO (US)

(21) Appl. No.: 19/313,481

(22) Filed: Aug. 28, 2025

Related U.S. Application Data
(60) Provisional application No. 63/688,181, filed on Aug. 28, 2024.

(51) Int. Cl. A61B 5/00; A61B 5/02

(52) U.S. Cl. CPC A61B 5/0077; A61B 5/02007; A61B 5/6824

(57) ABSTRACT

The present disclosure provides a biometric scanning system for generating an image of a user's vasculature. The biometric scanning system comprises a wrist-worn scanning device including NIR sensors for diffuse optical tomography, NIR LEDs, and an NIR camera configured to receive light emitted by the NIR LEDs after having passed through the user. The system processes vascular data collected by the NIR camera and/or the NIR sensors to generate a key corresponding to the user. In conjunction with access control systems, the biometric scanning system facilitates reliable user authentication without storing a root template of that user's vasculature.

### CROSS-REFERENCE TO RELATED APPLICATION

This application claims priority to U.S. Provisional Application No. 63/688,181, filed Aug. 28, 2024, which is hereby incorporated by reference in its entirety.

### GOVERNMENT RIGHTS

Embodiments of the present application were made in part with government support under Grant No. W911NF2120258 awarded by the United States Army Research Office. The government has certain rights.

### FIELD

The present disclosure relates to biometric identification systems, and more particularly to a multi-modal biometric system.

### BACKGROUND

Biometric identification systems have become increasingly prevalent in modern security applications. Traditional biometric modalities such as fingerprints, facial recognition, and iris scanning have been widely adopted due to their convenience and established recognition accuracy. However, these external biometric traits present inherent vulnerabilities that limit their effectiveness in high-security environments.

External biometric characteristics are susceptible to compromise (e.g., spoofing attacks.) For example, fingerprints can be lifted from surfaces and replicated using synthetic materials and facial recognition systems can be deceived through photographs, masks, digital manipulation techniques, or the like. These vulnerabilities create security risks in applications where robust authentication is necessary. The accessibility of external biometric traits also raises privacy concerns, as these characteristics can be captured covertly. This ease of acquisition makes it challenging to maintain the confidentiality of biometric templates and increases the risk of unauthorized surveillance or identity theft.

Current biometric template protection methods face challenges in balancing security with system performance. Traditional approaches often involve trade-offs between template security and matching accuracy, limiting their practical applicability. Advanced template protection schemes that can maintain high authentication performance while providing strong security guarantees remain an active area of research and development.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Aspects of the present disclosure permit secure personal identification and cryptographic key generation using a biometric scanning system for generating an image of a user's vasculature.

According to an aspect of the present disclosure, a biometric scanning system for generating an image of a user's vasculature is provided. The biometric scanning system comprises a strap configured to be secured around an extremity of the user, the strap comprising a plurality of near-infrared light emitting diode (LED) banks. The system includes an imaging unit comprising a near-infrared camera, the imaging unit configured to be releasably connected to the strap and positioned against the user's skin. The system further comprises a biometric scan processor and a memory, wherein the memory is a non-transitory computer-readable storage medium communicatively coupled to the processor and storing software including instructions which, when executed by the processor, enable the system to perform functions comprising selectively activating the LED banks and selectively activating the near-infrared camera to capture images of the user's vasculature as illuminated by the LED banks.

According to another aspect of the present disclosure, a unitary biometric scanning system for generating an image of a user's internal anatomy is provided. The unitary biometric scanning system comprises an imaging unit including a housing and a near-infrared camera located within the housing. The system includes a strap connected to the housing, the strap comprising a plurality of imaging clusters distributed along an inner face thereof, each imaging cluster comprising at least one near-infrared light emitting diode (LED) and at least one near-infrared sensor. The system further comprises a biometric scan processor and a memory, wherein the memory is a non-transitory computer-readable storage medium communicatively coupled to the processor and storing software including instructions which, when executed by the processor, enable the biometric scanning device to perform functions comprising selectively activating the imaging clusters to perform diffuse optical tomography measurements of tissue structures within the user and selectively activating the near-infrared camera to capture images of vascular patterns within the user.

According to another aspect of the present disclosure, a method of granting or denying access to a safeguarded entity is provided. The method comprises training a machine learning model using a plurality of vasculature images to generate, upon receiving an image of a person's vasculature, a unique and stable bit string associated with the person, wherein successive images of the person's vasculature will, when taken as input into the machine learning model, result in corresponding successive outputs of the same stable bit string. The method includes scanning, using a biometric scanning system, the person's vasculature to yield an image thereof, the scanning system comprising a biometric scan processor and a memory coupled thereto, the memory comprising the machine learning model. The method further comprises generating, by the machine learning model at the biometric scanning system, the stable bit string associated with the person, receiving, by an access control system configured for guarding the safeguarded entity, the stable bit string associated with the person, searching, by the access control system, a key list to determine if the stable bit string is present therein, and granting, by the access control system, the person access to the safeguarded entity if the stable bit string is present in the key list.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a top view of a first biometric scanning system.

FIG. 2 shows the first biometric scanning system being donned by a user.

FIG. 3 is a block diagram of the first biometric scanning system.

FIG. 4A is a top view of a second biometric scanning system.

FIG. 4B is a bottom view of the second biometric scanning system.

FIG. 5 shows the second biometric scanning system being donned by a user.

FIG. 6 is a block diagram of the second biometric scanning system.

FIG. 7 is a cross-section of an LED and thermal management system according to an embodiment.

FIG. 8 is a flow chart of an enrollment and authentication process according to an embodiment.

FIG. 9 is similar to FIG. 8, but includes visual aids.

FIG. 10A shows pseudo-code generally corresponding to the enrollment portion of the process shown at FIG. 8.

FIG. 10B shows pseudo-code generally corresponding to the authentication portion of the process shown at FIG. 8.

### DETAILED DESCRIPTION

The present disclosure will begin by describing the utility of vascular structures as biometric identifiers. Then, examination of the physical components and operational functionality of the biometric scanning system is presented with reference to first and second embodiments thereof. Description is also provided with respect to how the hardware and software components work together to create a secure authentication system. Further, cryptographic methods are presented for use in conjunction with the biometric scanning system.

### I. Vascular Structures as Biometric Identifiers

Vascular structures present unique advantages as biometric identifiers due to their inherent biological characteristics and accessibility through non-invasive imaging techniques. The vascular system exhibits substantial individual variation, with each person possessing a distinct pattern of blood vessels that remains largely stable throughout their lifetime; thus, it is challenging to replicate.

The vascular network accessible through near-infrared imaging is an abundant source of biometric information. Blood vessels absorb near-infrared light differently than surrounding tissue due to the optical properties of hemoglobin, creating distinct contrast patterns that can be captured and analyzed. These vascular patterns extend throughout the body and are well-suited for wrist-based imaging due to the relatively thin skin layer and high density of accessible blood vessels in this anatomical region. Those skilled in the art will recognize that other areas of the body may be used to similar effect (e.g., other portions of the arm, the legs, the neck, etc.).

Unlike external biometric traits such as fingerprints or facial features, vascular patterns are inherently protected from casual (e.g., discrete) observation and replication. The internal nature of these structures makes them resistant to spoofing attacks that commonly affect surface-based biometric systems (e.g., facial recognition systems). An attacker cannot simply photograph or mold vascular patterns as they would with fingerprints, nor can they easily manipulate the three-dimensional structure and optical properties of living tissue that create the unique signatures captured by diffuse optical tomography.

The temporal stability of vascular structures provides another advantage for biometric applications. While minor variations in blood flow and vessel dilation occur due to physiological factors such as temperature or exertion, the fundamental geometric structure of the vascular network remains consistent (e.g., the branching patterns, vessel diameters, etc.). Thus, biometric verification via vascular scanning provides long-term security solutions.

Diffuse optical tomography (DOT) enables the capture of volumetric information about vascular structures rather than simple surface patterns. DOT is a three-dimensional imaging technique which provides layered biometric information (e.g., vessel depth). The resulting data contains more entropy than traditional two-dimensional biometric modalities, supporting the generation of longer cryptographic keys and more robust authentication protocols.

The physiological nature of vascular patterns also provides inherent liveness detection capabilities. Living tissue exhibits characteristic optical properties related to blood oxygenation, flow dynamics, and cellular metabolism that are difficult to replicate artificially.

The pulsatile nature of blood flow creates temporal signatures that can be incorporated into the biometric matching process, providing additional security against presentation attacks using artificial materials or deceased tissue. This enables the scanning system to distinguish between living and deceased individuals, which can be used to prevent adversaries (e.g., military combatants) from using deceased individuals' anatomy in biometric verification.

The combination of multiple vascular imaging modalities within a single device further enhances the robustness of the biometric system. Different illumination patterns, wavelengths, and sensor configurations can capture complementary aspects of the vascular structure, creating a multi-modal biometric system that leverages the strengths of each imaging approach. This redundancy improves overall system reliability and provides multiple independent sources of biometric information for enhanced security protocols.

### II. The Biometric Scanning System

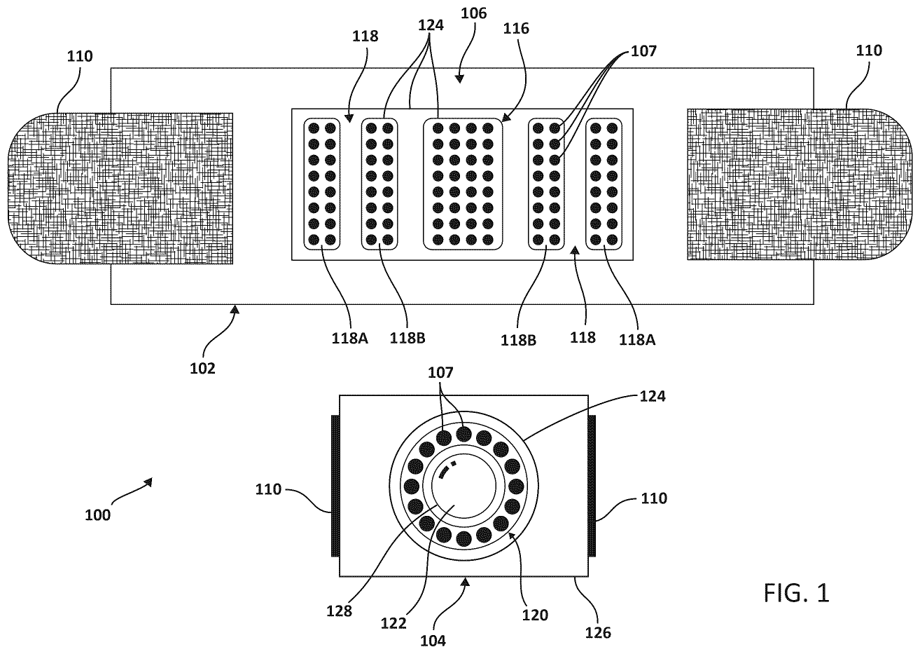

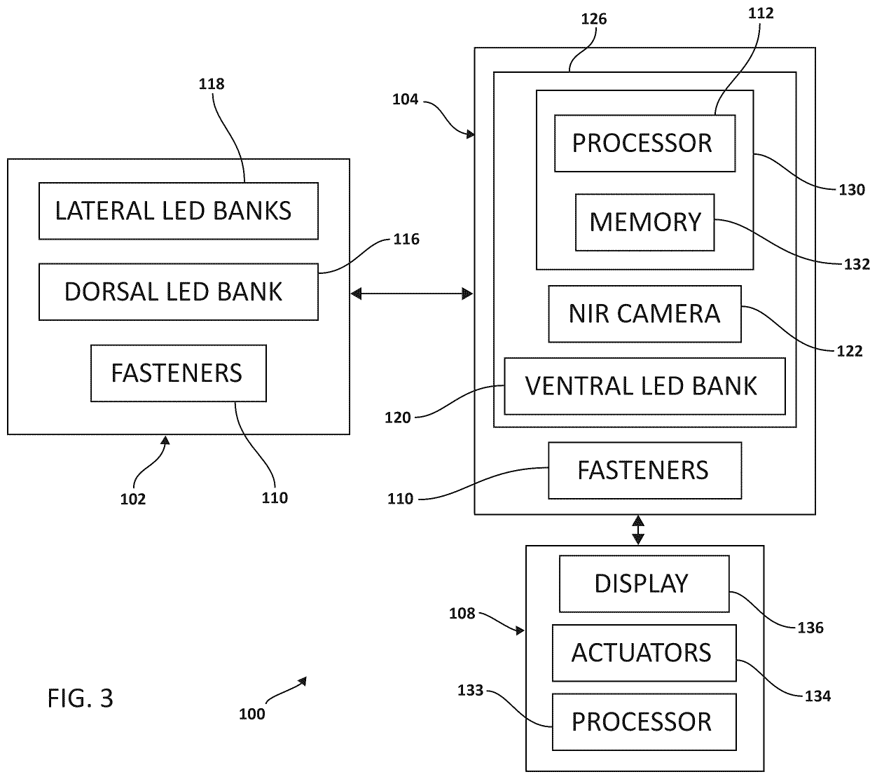

Referring to FIGS. 1-3, a first embodiment of a biometric scanning system (broadly, the scanning system, the biometric system, the system) in accordance with the present disclosure is generally indicated at reference number 100. The scanning system 100 broadly comprises a wrist strap 102, an imaging unit 104, a plurality of light emitting diode (LED) banks 106—each including a plurality of LEDs 107—and a user command module 108. The wrist strap 102 comprises most of the plurality LED banks 106, and is configured to flexibly conform to the shape of a user's wrist 109 while worn thereby. The wrist strap 102 and imaging unit 104 include fasteners 110 (e.g., hook and loop fasteners) configured to releasably adhere to one another, such that the wrist strap and imaging unit may be connected around the user's wrist 109. The LEDs 107 emit near-infrared light at wavelengths optimized for skin penetration and illumination of internal vascular structures, while the imaging unit 104 captures the light after it passes through the user's 114 tissue. A biometric scan processor 112 analyzes the captured light patterns to extract unique biometric features from the user's 114 internal tissue and vascular characteristics; specifically, in combination with an access control system (not shown), the system 100 provides secure biometric-based identity verification protocols which operate in the absence of a root biometric template. As will be explained in greater detail below, the entropic and unique nature of internal vascular structures as identified by the biometric scanning system 100, in combination with the identity verification protocols of the present disclosure, provides for improved security.

The wrist strap 102 (broadly, the strap) is designed to securely position the imaging unit 104 and LED banks 107 against the user's skin. The strap 102 incorporates a normally open mechanical switch (not shown) that closes only when properly secured around the wrist, providing continuous wear detection. This switch connects to the biometric scan processor 112, enabling immediate detection if the strap 102 becomes detached during operation. This continuous wear detection enhances security by preventing unauthorized access attempts when the device is removed from the legitimate user 114, and also improves reliability by ensuring that biometric scans are only attempted when the device is properly positioned for accurate data capture.

The mechanical switch also functions as part of an electrical interface between the strap 102 and the imaging unit 104, facilitating both power distribution and data communication throughout the system 100. When the strap 102 is properly secured and the mechanical switch closed, it may complete electrical pathways that enable the imaging unit 104 to deliver power to the LED banks 106 distributed throughout the strap 102. This electrical connection further establishes bidirectional data communication, allowing the biometric scan processor 112 within the imaging unit 104 to send control signals to individual LED banks 106 for selective activation and timing sequences, and to receive feedback data from the LED banks 106 (e.g., operational status information, temperature readings, light output measurements, etc.). The switch ensures that power delivery and data communication occur only when the device is properly positioned and secured.

The physical design of the strap 102 includes fasteners 110 on opposite ends thereof for releasably connecting to the imaging unit 104. In the illustrated embodiment, the fasteners 110 are hook-and-loop fasteners for enabling customizable adjustments for the user 114. In alternative embodiments, other fastening means may be used to secure the straps (e.g., buckles, snap fasteners, magnetic clasps, hook-and-loop fasteners, elastic bands, button closures, slide locks, pin-and-hole adjusters, etc.).

The strap 102 houses multiple LED banks 106 configured to independently illuminate in response to commands from the biometric scan processor 112, and perform individual functions therefor. The LED banks 106 are arranged in specific zones-dorsal (positioned on the back of the wrist 109), lateral (positioned on sides of the wrist), and ventral (positioned around the imaging unit 104 on the front of the wrist). Each of the LED banks 106 comprises a plurality of the near-infrared LEDs 107. In one aspect, these LEDs operate within a range of 600-1000 nm wavelength.

The dorsal LED bank 116 is positioned centrally on wrist strap 102 such that it aligns with the back of the wrist 109 when the scanning system 100 is worn. The dorsal LED bank 116 comprises many near-infrared LEDs 107 arranged to provide through-illumination of the wrist tissue (e.g., illumination of sufficient intensity to be detectable by the imaging unit 104 positioned opposite therefrom); as illustrated, this bank may include a higher number of LEDs compared to other illumination zones to compensate for optical attenuation resulting from wrist bones and tissue layers.

The LEDs 107 of the dorsal LED bank 116 are configured to emit light that penetrates through the wrist from the dorsal side, creating optical pathways that reveal deeper vascular structures that may not be visible through surface or lateral illumination alone. The positioning of this bank 116 enables the capture of transmitted light patterns that highlight three-dimensional vascular networks within the wrist 109 tissue. When activated, the dorsal LEDs 116 illuminate the tissue from behind, allowing the imaging unit 104 to capture light that has traveled through multiple tissue layers and vascular structures. Through-illumination provides enhanced contrast for deeper blood vessels while minimizing surface artifacts such as skin texture or superficial reflections. The dorsal bank 116 may be controlled independently by the biometric scan processor 112, enabling sequential activation patterns that allow for multi-exposure imaging and the creation of composite images that combine information from different illumination pathways.

The lateral LED banks 118 are positioned on the sides of the wrist strap 102 and comprise two separate illumination zones 118A and 118B to accommodate various wrist 109 sizes and shapes. Each lateral bank contains multiple near-infrared LEDs 107 arranged to provide side-illumination of the wrist 109 tissue, creating optical pathways that complement the dorsal and ventral illumination pathways. Lateral positioning enables illumination of vascular structures from angles that may reveal different aspects of the vascular network. The dual lateral bank configuration (118A and 118B) enables the system (e.g., the biometric scan processor 112) to dynamically select the optimal illumination zone based on the user's wrist size and the detection of light leakage, ensuring consistent image quality across different anatomical variations. More (e.g., three) or fewer (e.g., one) lateral LED banks 118 may be employed in alternative embodiments.

The lateral LED banks 118 operate at the same or similar wavelength range as other illumination zones, but may be configured with different power levels or activation patterns to optimize tissue penetration and reception by the imaging unit 104. As will be explained in greater detail below, during operation, the biometric scan processor 112 evaluates both the 118A and 118B banks through a light leakage detection algorithm that analyzes illumination uniformity and selects the configuration that minimizes unwanted light spillage around the wrist's 109 circumference. Adaptive selection ensures that lateral illumination provides maximum contrast for vascular structures, and may be applied with respect to any of the LED banks 106.

The ventral LED bank 120 is located at the imaging unit 104, and surrounds the NIR camera 122 itself. The ventral LED bank 120 is positioned concentrically around the NIR camera 122 within the imaging unit 104, creating a ring of illumination that provides incident lighting directly onto the ventral surface of the wrist 109; this configuration enables uniform illumination of the tissue surface immediately beneath the imaging unit 104. The close proximity of the ventral LED bank 120 to the NIR camera 122 allows for the capture of superficial vascular patterns and skin texture details that may not be visible through deeper illumination pathways; surface information complements deeper vascular data obtained through dorsal and lateral illumination, creating a more comprehensive biometric profile that combines both internal and external anatomical features. The ventral LED bank 120 may be configured with adjustable power levels to optimize balance between surface details and penetration depth, ensuring that both superficial and slightly subsurface vascular structures are adequately illuminated.

The LED banks 106 are employed together to create a pseudo-RGB composite image that encodes different tissue depths and optical pathways into a single-color photograph. In one embodiment, dorsal LED bank 116 data (e.g., visual data acquired via illumination of the wrist 109 via the dorsal LED bank) is mapped to a red channel; lateral LED bank 118 data (e.g., visual data acquired via illumination of the wrist via either or both of the lateral LED banks 118A, 118B, depending on optimal configuration) is mapped to a green channel; and ventral LED bank 120 data (e.g., visual data acquired via illumination of the wrist via the ventral LED bank) is mapped to a blue channel. This color-based image encoding transforms multiple grayscale captures from different illumination angles into a single color image that contains more biometric information than any individual capture alone. The resulting composite image may be used as an input for biometric feature extraction algorithms for leveraging the multi-channel data to identify unique vascular patterns for secure authentication and cryptographic key generation. In essence, the pseudo-RGB image is a two-dimensional image which contains data of similar information-density to a three-dimensional diffuse optical tomography scan. While red, green, and blue colors are employed in the presently referenced embodiment, those skilled in the art will recognize that alternative color combinations may be employed. While only three color channels are employed in the presently referenced embodiment, those skilled in the art will recognize that fewer color channels (e.g., two color channels) or more color channels (e.g., four color channels, five color channels, six color channels, seven color channels, eight color channels, nine color channels, ten color channels) may be employed.

Each of the LED banks 106 are individually sealed to prevent light leakage and maintain optical isolation between different illumination zones. The sealing is accomplished through sealing members 124 that create light-tight barriers around each LED bank 106, ensuring that illumination from one zone does not interfere with the optical pathways of adjacent zones. In some aspects, the sealing members 124 comprise rubber, silicone, or other flexible materials that conform to the contours of the wrist 109 to maintain an effective light barrier. Additionally or alternatively, all LED banks 106 may be collectively sealed together using a comprehensive sealing element that encompasses the entire illumination array. This protects against ambient light interference while maintaining the controlled illumination environment necessary for accurate biometric capture. The sealing members 124 may also create consistent contact pressure between the LEDs 107 and the user's 114 skin, further enhancing efficiency and reducing optical artifacts that could compromise image quality.

The imaging unit 104 comprises several integrated components configured to operate cooperatively to capture high-resolution vascular biometric data. A housing 126 serves as a protective enclosure for all internal components. The housing 126 is preferably constructed from durable materials (e.g., polymers, metals, etc.) and is contoured to maintain optimal positioning against the user's wrist 109.

Within the housing 126, the NIR camera 122 peers outwardly therefrom through an imaging port 128 defined at an inner face of the housing 126 (e.g., the side of the housing in contact with the user's wrist 109). The imaging port 128 may be spanned by a lens configured to protect the NIR camera 122 from damage or visual obfuscation which may be incurred via contact with the user's 114 skin (e.g., sweat) or from other sources (e.g., dust). The NIR camera 122 captures light emitted by the LED banks 106 after it passes through tissue. The ventral LED bank 120 surrounds the NIR camera 122, providing uniform illumination directly onto the skin to reveal superficial vascular patterns and enhance image contrast. In some aspects, the ventral LED bank 120 is of other geometries. A sealing member 124 creates a light-tight barrier between the imaging unit 104 and the user's skin, preventing ambient light interference and enabling reliable performance.

The imaging unit 104 also includes an electronics unit 130 comprising the biometric scan processor 112 and memory 132. The electronics unit 130 performs various computing/control functions used for image acquisition, processing, and biometric analysis. The biometric scan processor 112 executes functions for controlling LED illumination sequences (e.g., selectively activating the LED banks 106), selectively activating the near-infrared camera 122 to capture images of the user's 114 vasculature as illuminated by the LED banks 106, managing exposure settings, performing real-time image enhancement operations, generating high dynamic range images using perceptual exposure fusion, facilitating communication, etc. The memory 132 stores the operational software executed by the biometric scan processor 112, and may be configured for temporary storage of image data captured by the NIR camera 122. In the illustrated embodiment, the electronics unit 130 resides within the housing 126 for self-contained operation. Alternative configurations include an external electronics unit wherein processing occurs on a separate computing device connected to the imaging unit 104. For example, when implemented externally, a standard laptop or desktop computer may interface with the imaging unit 104 through secured data connections, providing processing capabilities while maintaining core imaging functionality.

The electronics unit 130 may include a wireless communications element (e.g., an antenna, not shown) configured to establish communication with external systems. This enables the scanning system 100 to transmit authentication results, biometric data, or cryptographic keys to remote access control systems, security networks, or other authorized devices without requiring physical connections. The wireless communications element may support various communication protocols including Wi-Fi, Bluetooth, near-field communication (NFC), or cellular networks, depending on the specific application requirements and security considerations. In some aspects, the wireless communications element may be configured to operate in secure communication modes that transmit encrypted data to prevent interception or unauthorized access during transmission. The integration of wireless communications enables system integration with existing security infrastructure, enabling the scanning system 100 to function as part of one or more larger authentication networks.

The illustrated embodiment includes a user command module 108 which is external to the imaging unit 104. The user command module 108 is generally configured to enable an operator (e.g., the user 114) to control functions of the scanning system 100. The user command module 108 houses a command processor 133, two actuators 134 (e.g., buttons) labeled “Enroll” and “Verify”, and a display 136 (e.g., an LED display, an OLED display). The display 136 is connected to the command processor 133 and provides operational feedback and status messages to guide the user 114 through the biometric scanning process. The user command module 108 includes a power management system which may be configured for both or either of internal or external power, enabling alternative portable and stationary use. The housing is constructed to provide protection for the internal components while maintaining accessibility to the actuators 134. When an operator presses either the “Enroll” or “Verify” button, the command processor 133 receives this input through initiates the corresponding scanning sequence, with real-time status updates communicated to the operator via the display 136.

The distribution of processing functions between the biometric scan processor 112 in the imaging unit 104 and the command processor 133 may be configured in various ways to optimize system performance and design requirements. In some aspects, processing operations may be entirely centralized at the command processor 133, with the imaging unit 104 functioning primarily as a data capture device that transmits raw sensor data to the external command module 108 for all computational tasks including image processing, biometric analysis, and system control. Conversely, the biometric scan processor 112 may handle all computational functions while the command module 108 serves merely as a user interface that relays commands and displays status information. In other configurations, processing tasks may be distributed between both processors, with the biometric scan processor 112 managing time-sensitive operations such as LED control sequences and initial image capture, while the command processor 133 handles more computationally intensive tasks such as biometric algorithms and data storage operations. This allows for optimization based on factors such as power consumption, processing speed requirements, and system complexity. In aspects which will be examined in greater detail later in the present disclosure, the user command module 108 may be integrated directly into the wrist-worn device itself, eliminating the need for an external command hub and resulting in a single processor configuration that manages all system functions.

The scanning system 100 employs diffuse optical tomography (DOT) inspired multi-path lighting to enable conventional NIR imaging devices to capture anisotropic optical properties of vascular tissue at different angles and depths. This increases the difficulty of performing physical and digital presentation attacks against the system 100. The system 100 also overcomes high scattering and extreme brightness gradients of illuminated tissue through multi-exposure high dynamic range (HDR) imaging. This enables the system 100 to capture detailed vascular patterns at various tissue depths that would otherwise be obscured by the optical properties of skin and surrounding tissues, thereby improving biometric accuracy and security. Additionally, as briefly discussed above, the system 100 creates pseudo-RGB images to similar effect. This yields clear, information-dense color images of the wrist for secure biometric applications.

Computational photography techniques, particularly HDR imaging and multi-path illumination, enhance biometric image quality by creating separate pathways for NIR light and representing those separate pathways within a two-dimensional image. By alternating illumination of the LED banks 106, the scanning system 100 produces effects similar to diffuse optical tomography at the camera level, enabling pseudo-RGB encoding of each pathway. Adaptive, multi-side NIR illumination fuses HDR images from different optical pathways into information-dense images that improve clarity and vascular visibility at various depths, while making replication increasingly difficult for attackers. These information-dense images enable improved performance by software systems in identifying vascular characteristics and the associated individual.

Having described the physical components of the first embodiment of the biometric scanning system 100, a detailed description of its use will be provided via an example scanning procedure.

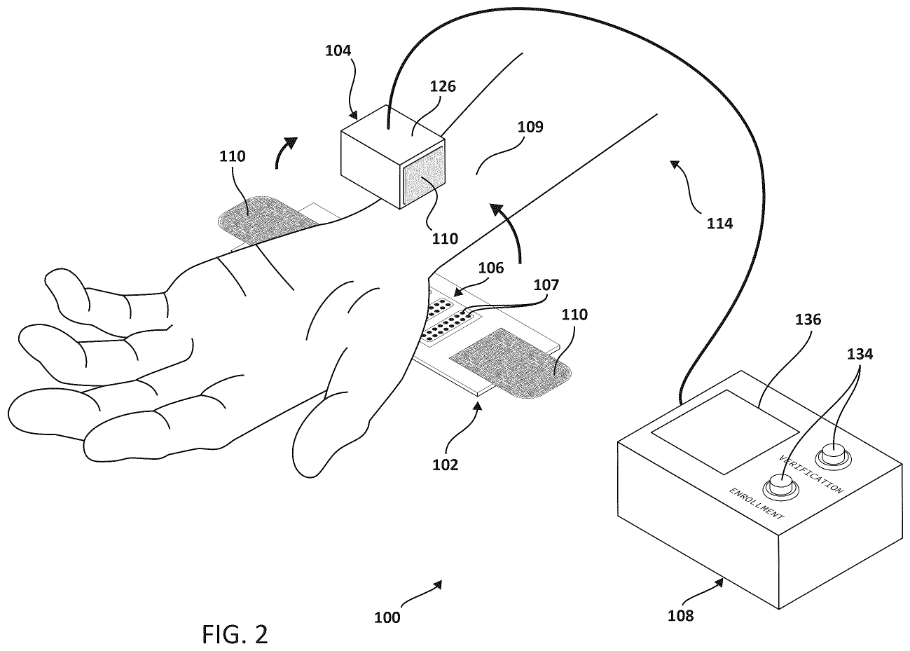

To begin a scanning process according to an embodiment, the user 114 (e.g., subject) secures the strap 102 and imaging unit 104 around their wrist 109 as shown at FIG. 2. Once properly positioned and activated (e.g., using the user command module 108), a calibration sequence is initiated by sequentially activating lateral illumination zones 118A and 118B. For each zone, a low-exposure reference image is captured and analyzed using a light leakage detection algorithm. This algorithm divides each image into three vertical regions (left, center, and right, although more vertical regions may be employed), calculates the average intensity of each region, and determines a leakage ratio by comparing intensity at the side regions (left and right) to intensity at the center. Based on this analysis, the optimal lateral illumination configuration for the user's 114 specific wrist size is selected, thereby mitigating light leakage.

After selecting an optimal lateral LED bank 118, a multi-zone image capture sequence is executed. This begins with “through” image capture using the dorsal LED bank 116. A dorsal image stack is collected, comprising five images captured during illumination of the dorsal LED bank 116 at different exposure levels. An auto-exposure reference point is determined by the biometric scan processor 112 and followed by four additional exposures (e.g., 5,000 μs, 50,000 μs, 150,000 μs, and 350,000 μs). In certain aspects, more or fewer images may be captured at this sequence, and at different exposures. The same is true for all image stacks described below.

Next, similar functions are performed using the lateral LED banks 118 by activating the selected lateral LED bank (either or both of 118A or 118B based on the calibration results) and capturing a lateral image stack comprising a plurality of lateral images taken at various exposures. This follows the same exposure pattern used for the dorsal image stack to provide complementary information about mid-depth vascular structures from a different angle.

The ventral LED bank 120 is then activated to illuminate the front of the wrist, and a ventral image stack comprising a plurality of ventral images is collected. This follows the same exposure pattern used for the dorsal and lateral image stacks. Throughout these image captures sequences, gain (e.g., signal amplification) is kept constant.

Those skilled in the art will recognize that the order in which the image stacks are captured may be varied without departing from the scope of the present disclosure. While the described embodiment captures the dorsal image stack first, followed by the lateral image stack, and then the ventral image stack, alternative sequences may be employed based on specific application requirements, processing considerations, or system optimization needs. In some aspects, the lateral image stack may be captured first, or the ventral image stack may precede the dorsal capture sequence. The system may also be configured to capture image stacks in parallel or in overlapping sequences to reduce total acquisition time while maintaining image quality and system performance.

Once all fifteen images (five per illumination zone) are captured, each image stack is processed into a single HDR image (e.g., a dorsal HDR image from the dorsal image stack, a lateral HDR image from the lateral image stack, and a ventral HDR image from the ventral image stack). In some aspects, the biometric scan processor 112 is configured to generate these HDR images using perceptual exposure fusion. HDR imaging techniques emphasize well-exposed pixels with strong local contrast, creating three distinct HDR images-one for each illumination zone. Each resulting HDR image captures different aspects of the wrist's vascular structure: the dorsal HDR image reveals deeper vascular structures, the lateral HDR image highlights mid-depth features with enhanced edge contrast, and the ventral HDR image captures superficial vasculature and skin texture details.

Following HDR creation, quality assessment may be performed on each HDR image. For exposure quality evaluation, the Center of Mass (COM) of each image's histogram is calculated (see equation 1 below). HDR images which fall outside a given range may be flagged and/or discarded. For example, HDR images with COM below 20 may be flagged as underexposed, while those above 180 may be flagged as overexposed; these values may be adjusted to varied effect. If quality issues are detected, recapture may be automatically initiated with adjusted exposure values. If quality issues persist after multiple retries, the best available image from that LED bank's image stack is selected based on exposure metrics and Shannon Entropy calculations. This promotes optimal image quality despite variable conditions such as hand placement or ambient light interference.

$$CoM = \frac{{\sum }_{i = 0}^{255} i \cdot h ( i )}{{\sum }_{i = 0}^{255} h ( i )} \qquad ( 1 )$$

After validating all HDR images, a composite pseudo-RGB image is generated therefrom. This comprises assigning the dorsal HDR image to a red channel, the lateral HDR image to a green channel, and the ventral HDR image to a blue channel. Each channel may be individually enhanced using Contrast Limited Adaptive Histogram Equalization (CLAHE). This technique divides each channel into tiles, applies histogram equalization locally, and clips histogram peaks to prevent over-enhancement while improving local contrast. The resulting pseudo-RGB image combines information from all three illumination zones, creating a comprehensive representation of the wrist's 109 vascular structures at multiple depths.

The presence of body hair on the wrist 109 can interfere with accurate biometric scanning by obscuring vascular patterns and creating unwanted artifacts in captured images. To ensure optimal image quality and reliable biometric feature extraction, an automated hair detection and removal process may be implemented.

In this process, the image (e.g., the pseudo-RGB image) is analyzed to determine if hair artifacts are present. This analysis begins by applying a “black-hat” morphological filter to highlight line-like features typical of hair strands. Statistical features are then extracted including mean, standard deviation, maximum intensity, and high percentile values, which are fed into a Random Forest classifier trained to identify hair presence.

If the presence of body hair is detected, the removal process is initiated. The pseudo-RGB image is first converted to grayscale, and black-hat morphological operations are applied in both vertical and horizontal directions to highlight hair-like structures. These results are combined and binarized using adaptive Gaussian thresholding to generate a hair mask. The mask undergoes refinement through morphological opening and closing operations, followed by connected component analysis to remove noise and small artifacts. An inpainting algorithm is then applied to reconstruct the hair-occluded regions identified by the mask (e.g., to generate a hair-removed pseudo-RGB image). The result is smoothed using an edge-preserving filter and blended back into the RGB image using Gaussian mask blending.

After hair removal, the image is cropped to the specific region of interest containing the most relevant vascular features, resulting in a processed image optimized for biometric analysis. Depending on initial selection (enrollment or verification), the processed image (e.g., the hair-removed pseudo-RGB image) is routed to the appropriate biometric pipeline. For enrollment, image features are extracted and stored. For verification, the extracted features are used to determine identity.

As will be explained in greater detail in the section covering software implementation, a key is generated during verification which is hashed and compared with a stored digest. This returns only a match or no-match flag while ensuring the key itself remains protected. No biometric templates (e.g., “original” scans of vasculature) are stored.

The multi-zone HDR technique described produces improved visibility of both superficial and deep vascular features by mitigating uneven exposure issues common in wrist imaging, while the pseudo-RGB encoding preserves and enhances features at multiple tissue depths. The resulting images yield detailed vascular patterns that enable accurate biometric matching while resisting spoofing attempts. In summary, the image capture and processing workflow of the present example aspect consists of: (1) wristband placement and lateral illumination zone selection, (2) sequential multi-exposure capture from the LED banks 106, (3) HDR fusion of each exposure stack, (4) quality assessment and potential recapture, (5) pseudo-RGB image construction and CLAHE enhancement, (6) hair detection and removal if necessary, (7) region of interest cropping, and (8) secure biometric processing for enrollment or verification.

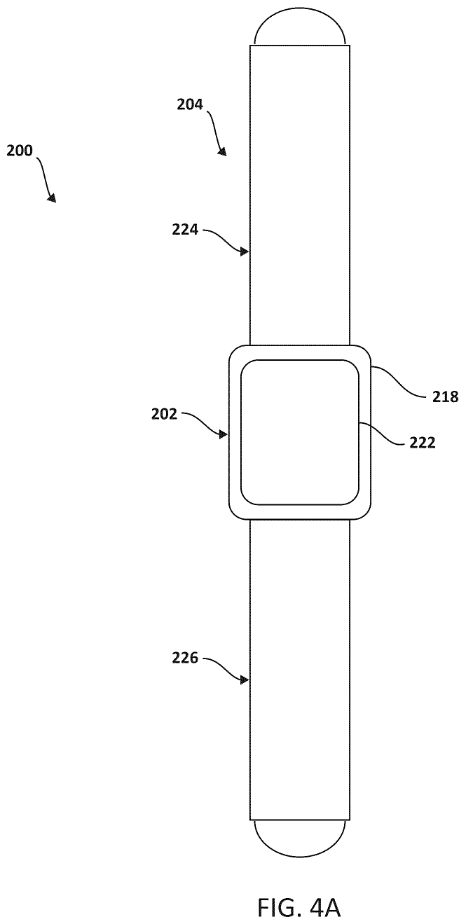

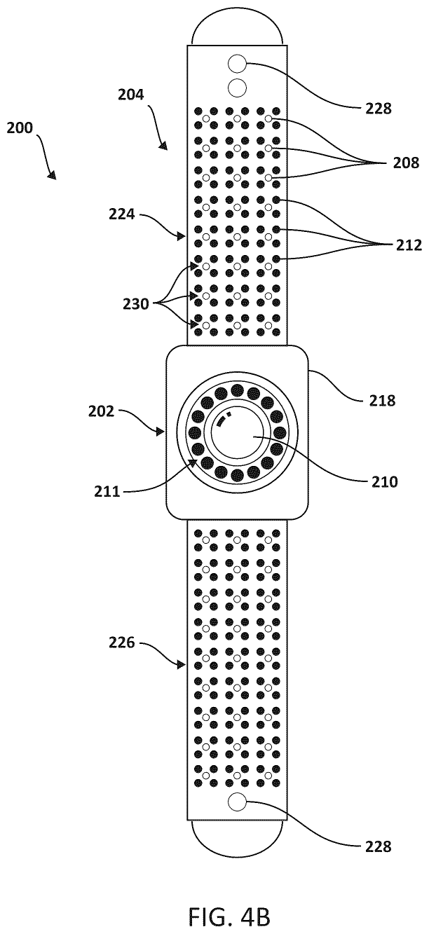

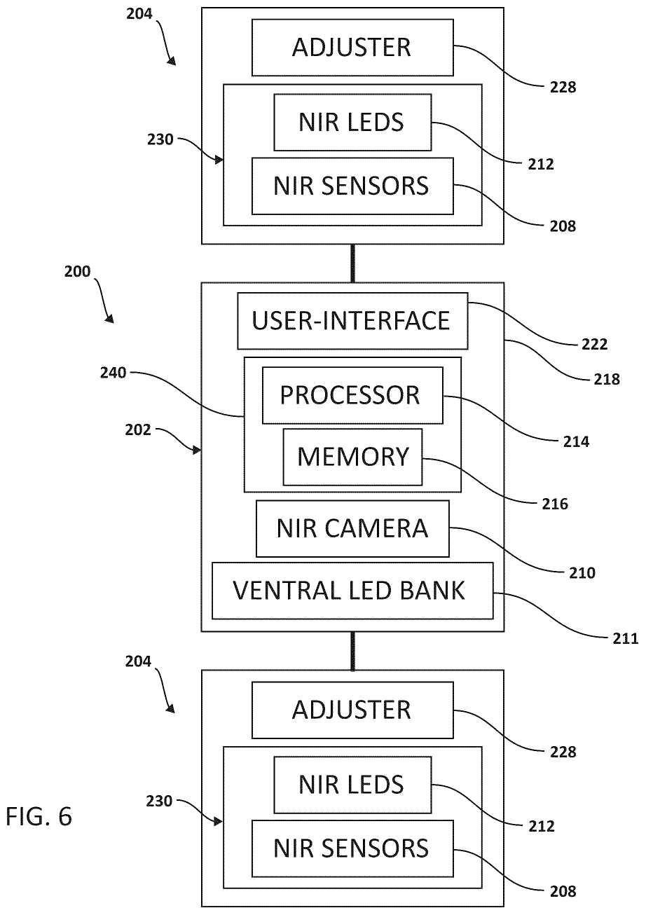

Referring to FIGS. 4A-6, a second embodiment of the biometric scanning system is generally indicated at reference number 200. This embodiment presents a unitary wrist-worn device which integrates all system components into a single unit. Unlike the scanning system's 100 three-piece design, the unitary scanning system 200 consolidates the imaging unit 202, wrist strap 204 (broadly, strap), and user command module into a cohesive wearable device. Additionally, instead of simulating three-dimensional scans as performed using the scanning system 100, the unitary scanning system 200 employs authentic diffuse optical tomography via NIR sensors 208 in conjunction with an NIR camera 210 and NIR LEDs 212 to provide dual functionality.

The imaging unit 202 houses the NIR camera 210, ventral LED bank 211, processor 214, memory 216, a power source (e.g., a battery, not shown), a communications element (e.g., an antenna, not shown), and other electronics components necessary for image capture and processing operations. The imaging unit 202 captures light emitted by the LEDs 212 after it passes through tissue, processes images, and generates biometric keys for enrollment and/or authentication processes. The housing 218 maintains optimal positioning against the user's 220 wrist through the integrated strap design while protecting internal components from environmental factors.

Similarly to the first embodiment 100, the NIR camera 210 and ventral LED bank 211 of the unitary scanning system 200 operate cooperatively to capture high-resolution vascular biometric data from the ventral surface of the user's 220 wrist. The ventral LED bank 211 is positioned concentrically around the NIR camera 210 within/on the imaging unit 202, creating a ring of near-infrared illumination that provides incident lighting directly onto the tissue surface immediately beneath the device. This enables uniform illumination of superficial vascular patterns and skin texture details, while the NIR camera 210 simultaneously captures the reflected and scattered light patterns after they interact with the user's 220 tissue structures. The ventral LED bank 211 may be configured with adjustable power levels to optimize the balance between surface detail capture and penetration depth, ensuring that both superficial and slightly subsurface vascular structures are adequately illuminated for detection by the NIR camera 210. Together, these components generate detailed imagery of the ventral vascular network that complements the deeper tissue measurements obtained through the distributed NIR sensors 208, creating a multi-modal biometric capture system that combines both surface and volumetric anatomical information.

The user command module is incorporated directly into the imaging unit 202. This creates a smartwatch-like interface 222 that eliminates the need for external command hardware while providing user 220 control. The integrated user interface 222 enables users to initiate enrollment or authentication processes, view system status, and access device settings without requiring separate command hardware.

The user interface 222 may include touch-sensitive controls, physical buttons, or gesture recognition capabilities that allow users to navigate through system functions. The processor 214 and user-interface 222 may be configured to provide real-time feedback during scanning operations, showing capture progress, image quality assessments, and authentication results. Status indicators may inform users of device readiness, battery levels, connectivity status, and operational issues.

The integrated design enables continuous operation modes where the device can perform periodic authentication verification while worn. Power management systems may be incorporated to optimize battery consumption by adjusting scanning frequency and processing intensity based on user 220 activity and security requirements. The consolidated architecture supports wireless communication protocols that enable the system 200 to interface with access control systems.

For example, the scanning system 200 may be integrated with access control systems to provide automated authentication for safeguarded entities such as secure facilities, weapon systems, or restricted areas. In this implementation, a machine learning model is trained using a plurality of vasculature images from authorized personnel to generate unique and stable bit strings that serve as cryptographic keys for each individual. As will be explained in greater detail in later sections, the training process ensures that successive scans of the same person's 220 vasculature will consistently produce the same stable bit string. During operation, when a person approaches a safeguarded entity, the biometric scanning system 200 captures an image of their vasculature and processes it through the trained machine learning model to generate their associated stable bit string. This bit string is then transmitted to the access control system guarding the safeguarded entity, which searches a pre-populated key list containing the stable bit strings of all authorized personnel. If the generated bit string matches an entry in the key list, the access control system grants the person access to the safeguarded entity. This approach enables authentication where authorized personnel can gain access simply by wearing the biometric scanning device 200, while the system 200 automatically handles the capture, processing, and verification of their vascular biometric data without requiring manual credential presentation or explicit user 220 actions for each authentication event.

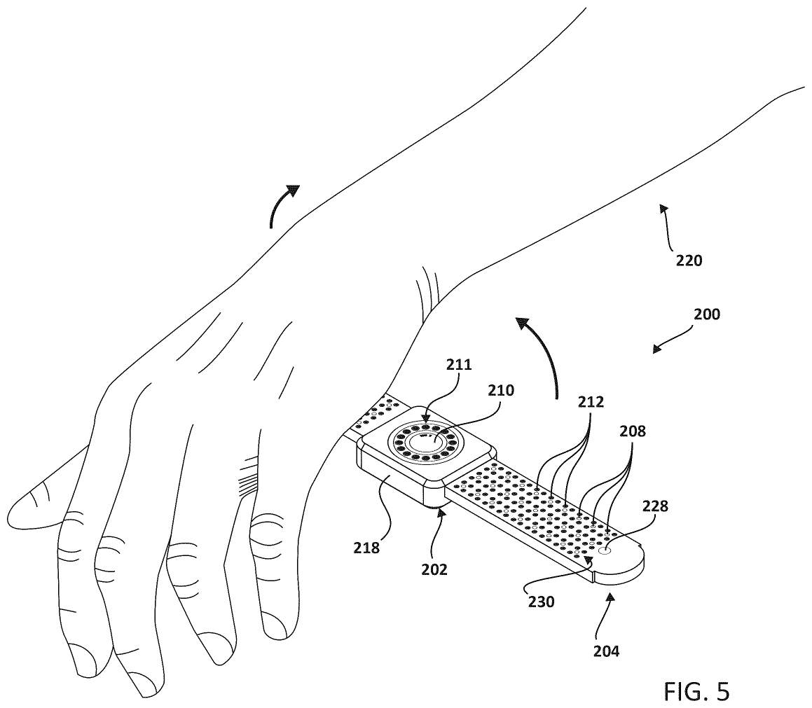

The wrist strap 204 of the unitary scanning system 200 includes a first strap portion 224 and second strap portion 226 extending outwardly from the housing 218 of the imaging unit 202 on opposing sides thereof. The first and second strap portions 224, 226 are configured to wrap around the user's 220 wrist and releasably connect to secure the system thereto, as shown at FIG. 5. The strap portions 224, 226 are constructed from a flexible material that conforms to various wrist sizes while maintaining sufficient tension to ensure stable positioning of the imaging components against the skin. Each strap portion includes adjustment mechanisms 228 to accommodate different user 220 profiles, and the releasable connection is designed to withstand user 220 activity without accidental detachment. An electrical connection bridges the strap 204 and the imaging unit 202 such that power and data may be exchanged therebetween.

The strap 204 incorporates a plurality of imaging clusters 230 distributed uniformly along its inner face (e.g., to form a planar LED array). Each cluster 230 comprises at least one NIR LED 212 and at least one NIR sensor 208. The illustrated embodiment comprises four NIR LEDs 212 arranged in a square configuration surrounding a centrally positioned NIR sensor 208. The unitary scanning system 200 includes dual-modality biometric capture, utilizing the NIR camera 210 and ventral LED bank 211 for high-resolution imaging of superficial vascular patterns while simultaneously employing the distributed NIR sensors 208 for DOT measurements of deeper tissue structures. Power management protocols and thermal dissipation features may be employed to accommodate LED 212 density and continuous operation requirements of the wearable form factor.

The imaging clusters 230 operate according to diffuse optical tomography principles, wherein each cluster functions as a source-detector pair capable of measuring light attenuation and scattering through tissue at various depths. The NIR LEDs 212 in each cluster emit coded light pulses (e.g., using Barker sequences) to achieve high signal-to-noise ratios while maintaining ultra-low power consumption. Coded illumination enables reduce instantaneous power requirements compared to traditional continuous wave illumination, thereby extending battery life and minimizing thermal effects on the user's 220 skin.

The system implements programmable illumination patterns where LED 212 clusters can be selectively activated in predefined sequences to perform both local and global DOT measurements. Local measurements focus on tissue properties immediately beneath individual clusters, while global measurements utilize multiple clusters simultaneously to create optical pathways that traverse larger tissue volumes. This flexibility enables the system to adapt its scanning depth and resolution based on biometric requirements and user 220 anatomy.

DOT measurements capture information about tissue optical properties including absorption and scattering coefficients, which may vary based on blood oxygenation levels, tissue density, and vascular architecture. These measurements provide metabolic information that complements the structural vascular data obtained through conventional NIR imaging, creating a multi-dimensional biometric signature.

In one aspect, power management in the unitary scanning system 200 utilizes on-demand scanning protocols wherein full biometric captures occur only during initial authentication or when the device detects removal from the user's 220 wrist. Between these events, the system 200 maintains authentication state through periodic low-power verification scans that consume minimal battery resources. The integrated battery system supports both active scanning operations and standby monitoring, with power distribution managed through intelligent switching circuits that optimize energy allocation based on operational requirements.

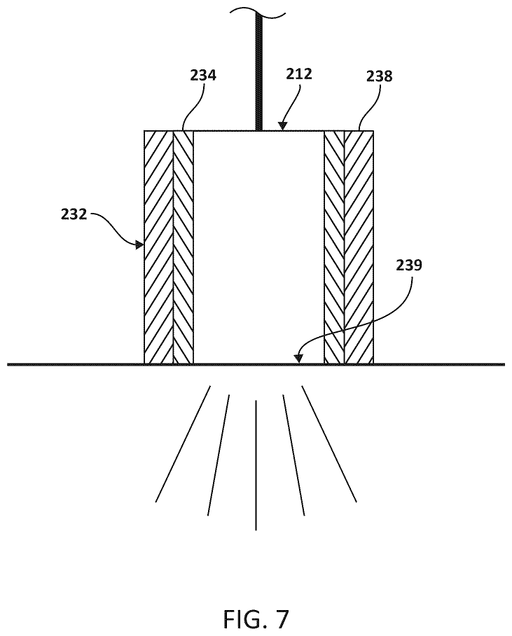

Thermal management may be necessary due to the increased component density (e.g., LED 212 density) and prolonged operation. The unitary scanning system 200 may incorporate a heat dissipation structure 232 surrounding each LED 212 to prevent skin irritation and ensure user 220 comfort during extended wear. As shown at FIG. 7, LEDs 212 may be encapsulated within a layered thermal management system. An inner layer 234 comprises material which is electrically insulating and thermally conductive (e.g., Aluminum Oxide (Al₂O₃)). The inner layer 234 is positioned adjacent to the LED's semiconductor junction for optimal performance. An outer layer 238 comprises a thermally conductive material and radially encompasses both the LED 212 and the inner layer 234. The inner layer 234 creates a thermally conductive bridge between the heated region of the diode and the outer layer 238 that efficiently distributes thermal energy. The outer layer 238 extends beyond the immediate LED area, maximizing surface area available for heat dissipation to the ambient environment. This architecture creates multiple pathways for heat to escape from the LED junction, preventing localized hot spots at the skin-device interface 239. The thermal management system may be replicated for each LED 212 throughout the wrist strap 204 to promote consistent performance and user 220 comfort.

The unitary scanning system 200 allows the wearer to unlock physical entrances, enable weapon systems, or communicate with identification friend-or-foe transponders without requiring explicit user 220 actions for each authentication event. For example, in a military context, a soldier wearing the device could approach a secure facility and gain access automatically as the wristband communicates with the entry system, verifying both the soldier's identity and authorization level without requiring manual credential presentation. Similarly, when the soldier picks up a weapon system equipped with compatible authentication technology, the weapon could automatically activate only when held by an authorized user 220, preventing unauthorized use if the weapon is lost or captured.

In civilian applications, the technology enables reduced friction access to facilities where the user 220 could pass through security checkpoints without stopping to present credentials, with the wristband transmitting the necessary authentication data to nearby readers in access control systems. For example, healthcare professionals could gain immediate access to medication dispensing systems or restricted hospital areas based on their biometric verification, reducing authentication friction during emergency situations.

The unified processing architecture in the unitary scanning system 200 consolidates all computational functions within a single electronics unit 240. In the illustrated embodiment, this includes a “system-on-chip” platform with a CPU, GPU, and neural processing unit capabilities. This enables real-time execution of complex software functions (e.g., deep learning models for biometric feature extraction) while maintaining a secure execution environment for cryptographic operations. The processing system can perform biometric matching, template protection, and private key generation entirely within the wearable device, eliminating dependencies on external computing resources.

While the multi-piece scanning system 100 and unitary scanning system 200 differ in their physical architecture and component distribution, both systems fundamentally serve a shared purpose of capturing and processing high-resolution vascular biometric data through near-infrared imaging techniques. The first embodiment achieves this through a modular three-piece design (wrist strap 102, imaging unit 104, and user command module 108) with external processing capabilities, while the second embodiment integrates all functionality within a unified wearable platform while also providing authentic DOT scanning abilities. Both systems 100, 200 generate detailed images that encode multi-depth vascular information suitable for secure biometric analysis.

The design principles and components described in all implementations/embodiments may be interchanged and combined in various configurations without departing from the scope of the present disclosure.

Having established the image acquisition and processing methodologies that enable both embodiments to produce vascular biometric data, the disclosure now turns to the cryptographic framework that converts these captured vascular patterns into authentication credentials and cryptographic keys.

### III. Template-Free Biometric Verification

The following sections of this disclosure present software and cryptographic methodologies that transform the captured vascular biometric data into keys capable of being verified without reference to a root template. This begins with an explanation of template-free biometric verification principles, followed by enrollment and authentication procedures.

The “template” is the root digital representation of one's biometric characteristics. Templates serve as reference patterns against which, in traditional security frameworks, future biometric samples are compared during authentication attempts. Biometric templates present security vulnerabilities because they represent permanent, unchangeable biological characteristics of individuals. Unlike passwords or cryptographic keys that can be revoked and replaced when compromised, biometric traits such as fingerprints, iris patterns, or vascular structures cannot be altered or reissued. Thus, their preservation is of elevated concern.

To facilitate enrollment and authentication processes without storing an individual's biometric template, a deep learning matcher that operates in a latent feature space is employed (e.g., a feature space wherein a compressed representation of biometric data is preserved, the compressed representation preserving essential features for informing machine learning algorithms). Rather than attempting to reconstruct complete images from the multi-path absorptions and reflections of the NIR light array—a computationally intensive task-biometric features are derived directly from the scanner readouts in a latent, pre-image space. This reduces computational loads with respect to classification, accelerating the authentication process and minimizing power consumption, which is beneficial for portable field implementations (e.g., in conjunction with the unitary scanning system 200).

A neural architecture search (NAS) is employed to find optimal deep learning structures for vasculature and DOT matching. This automatically adapts to variations in biometric presentation, and can be combined with one/few-shot learning for robust classification of vascular structures and/or DOT signatures, enabling rapid user enrollments (e.g., in field conditions). Various representation learning approaches may be employed (e.g., synthesized negative hard mining, autoencoders, Siamese networks with triplet loss, deep convolutional neural network structures with Additive Angular Margin Loss (ArcFace), etc.).

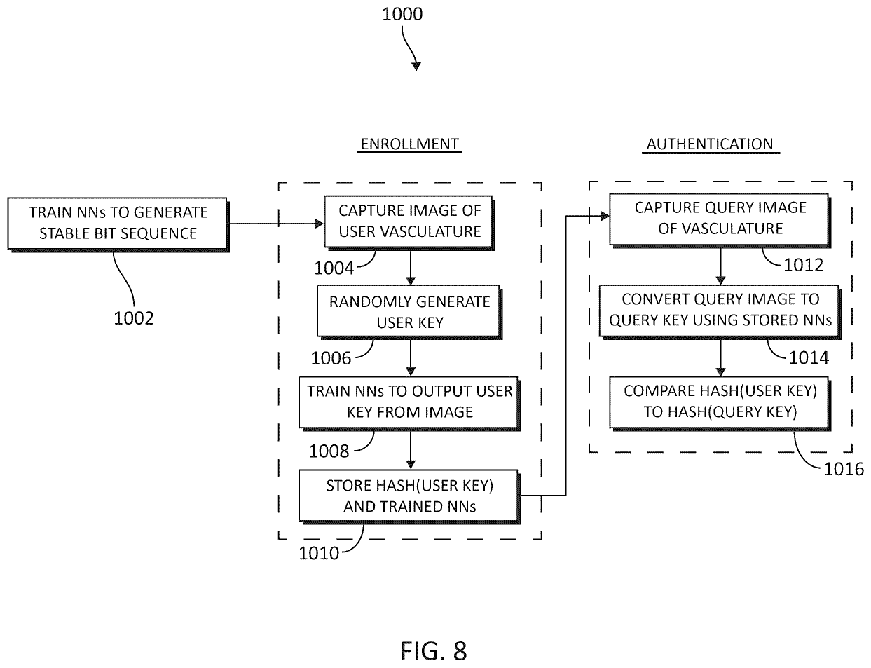

Referring to FIG. 8, a template-free biometric enrollment and authentication process is illustrated as a flowchart, and is generally indicated at reference number 1000. Prior to enrollment, at operation 1002, a bank of shallow neural networks (NNs) may be trained to generate stable bit sequences from biometric templates during the enrollment. Each NN corresponds to a specific bit position in the target binary sequence, collectively enabling the transformation of high-dimensional biometric features into reproducible cryptographic keys. Other binary classifiers (e.g., support vector machines) or combinations thereof may be used to build the private-key-generating classifier bank (e.g., in place of or in combination with the bank of shallow neural networks). This will described in greater detail at section IV of the present disclosure.

### III.A. Enrollment

Enrollment begins at operation 1004 with image capture of the user's vasculature. Multi-angle near-infrared illumination captures internal vascular structures through the scanning systems described previously (e.g., systems 100 and 200). The resulting biometric template serves as the foundation for subsequent authentication attempts, but is never stored in its raw form, thereby enhancing security against template compromise.

At operation 1006, a random user key is generated as a binary bit sequence. Variable key lengths may be used (32 bits, 64 bits, 128 bits, 256 bits, etc.) to accommodate different security requirements, with longer sequences generally providing enhanced cryptographic strength. This key functions as the target output for the neural network training process rather than being directly stored.

At operation 1008, the neural networks are trained to map the user's vascular biometric features to the generated user key. Each bit in the sequence is assigned to a dedicated neural network trained using the user's biometric features as positive samples and synthesized impostor features as negative samples. This enables the networks to produce consistent outputs despite minor variations in subsequent scans, addressing the inherent variability in biometric measurements while maintaining discriminative power.

At operation 1010, only a cryptographic hash (e.g., SHA-256) of the user key is stored, along with the trained neural networks. This ensures that neither the raw biometric template nor the actual key is retained, providing strong protection against both template reconstruction attacks and key compromise.

### III.B. Authentication

Authentication begins at operation 1012 with the capture of a query vascular image from a subject seeking verification. This is similar to operation 1004.

At operation 1014, the stored neural networks process the query image to generate a query key. Each neural network independently predicts one bit of the binary sequence, with the outputs being concatenated to form the complete query key. If the subject is the legitimate enrolled user, the neural networks will reproduce the original user key; otherwise, they will generate a different bit sequence.

At operation 1016, the same cryptographic hash function employed at operation 1010 is applied to the query key; HASH (query key) and HASH (user key) are then compared. The subject is authenticated as the original user only when HASH (query key)=HASH (user key), indicating a match between the query key and the original user key. The cryptographic properties of the hash function ensure that even a single bit difference between keys produces different hash values, providing strong security against partial matches or near-miss attacks.

This template-free authentication framework achieves the security properties of non-invertibility, revocability, and unlinkability while maintaining biometric accuracy. By eliminating stored templates and implementing one-way transformation through neural networks and cryptographic hashing, the system prevents reconstruction of original biometric data even if the stored components are compromised.

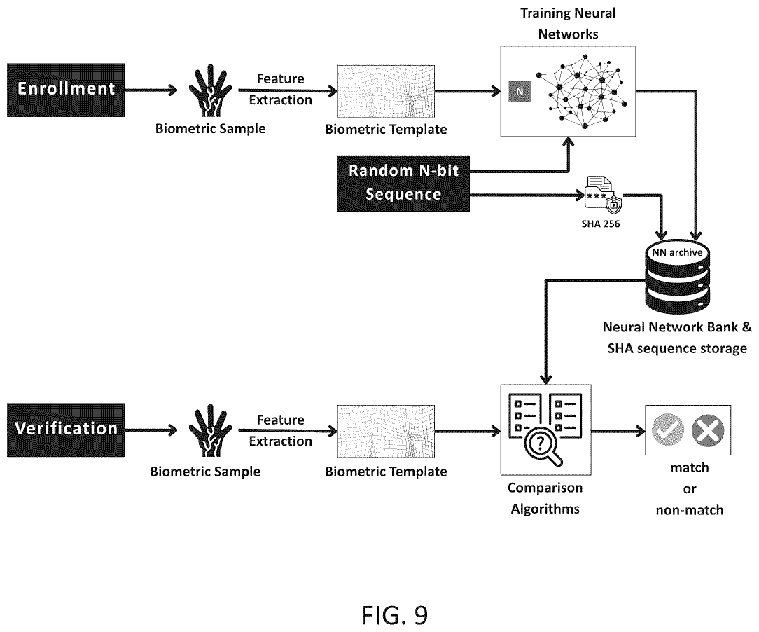

FIG. 9 is similar to FIG. 8, but shows the enrollment and authentication process 1000 with visual aids.

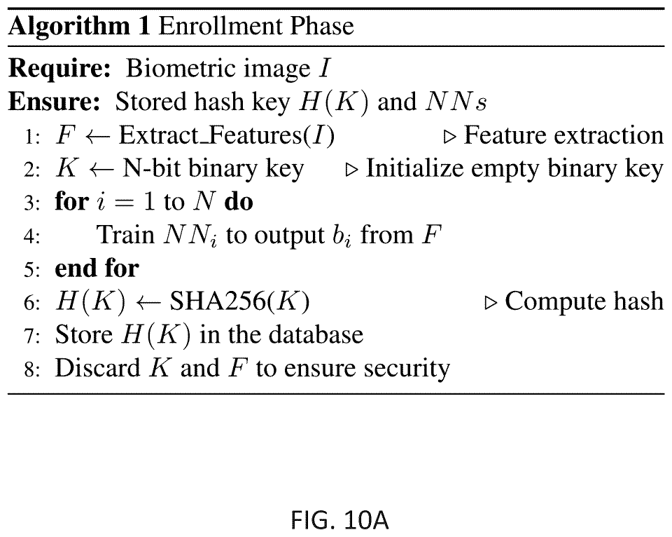

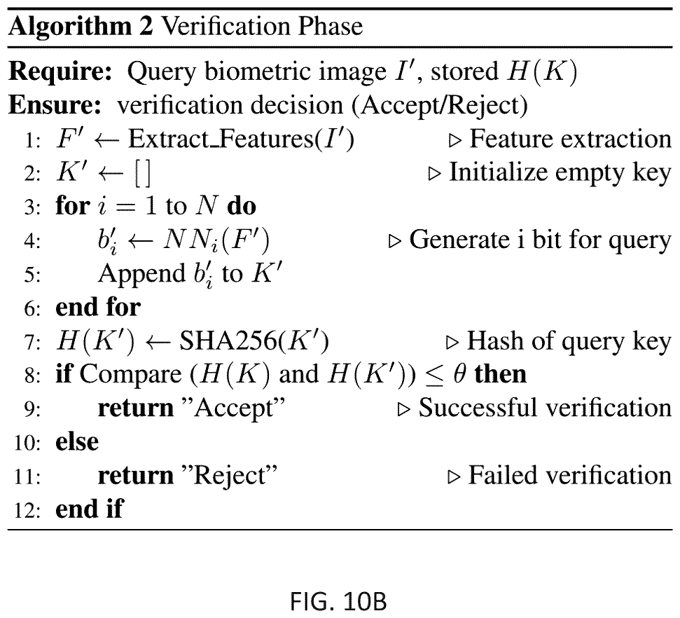

FIGS. 10A and 10B show pseudo-code corresponding to the enrollment and authentication (verification) portions of the process 1000, respectively.

IV. Biometric Key Generation from Vascular Data

Having described the enrollment and authentication process 1000, the manner in which biometric templates are converted into stable bit strings (e.g., keys) will now be examined in greater detail.

Biometric key generation begins with feature extraction from the captured vascular data (e.g., as captured by scanning systems 100 and 200). A convolutional neural network (CNN) architecture (e.g., ResNet-50) is employed for feature extraction. The network may be pre-trained on large-scale datasets (e.g., at operation 1002 of the process 1000 described above) and subsequently fine-tuned using vascular biometric data to optimize feature discrimination. During fine-tuning, specialized loss functions (e.g., additive angular margin loss) may be applied to maximize angular distances between different subjects in the feature space, thereby enhancing inter-class separability while maintaining intra-class compactness.

Feature extraction may utilize deeper layers of the neural network. For example, a ‘flatten’ layer, which transforms spatial dimensions of input data into a one-dimensional array containing detailed features from the vascular patterns, may be employed. Extracted features typically comprise high-dimensional vectors (e.g., 512 or 2048 dimensions) that capture the unique characteristics of the subject's vascular structure.

To manage computational complexity and improve processing efficiency, dimensionality reduction techniques may be applied to the extracted features. Principal Component Analysis (PCA) may be employed to transform high-dimensional feature vectors into lower-dimensional subspaces while preserving discriminative information. In some aspects, components that collectively explain a predetermined percentage of total variance (e.g., 95%) may be retained to ensure efficiency while maintaining biometric accuracy.

The conversion from continuous feature vectors to stable binary sequences presents unique challenges due to the inherent variability in biometric measurements. Traditional cryptographic hash functions are unsuitable for this purpose because minor variations in input features-which naturally occur between different scans of the same individual—can cause dramatic changes in the output hash due to the avalanche effect. Specialized deep biometric hashing techniques may be employed to overcome these challenges. These methods may utilize learnable binary hash mappers that are trained to produce consistent binary outputs despite minor variations in input features. The deep hash generation process comprises training a bank of binary classifiers, wherein each classifier corresponds to a specific bit position in the target binary sequence.

In some aspects (e.g., the enrollment and authentication process 1000 described above), binary key generation is accomplished using a bank of shallow neural networks, each configured to generate a single bit of the target sequence. Each neural network in the bank may comprise an input layer matching the dimensionality of the extracted features, one or more hidden layers with a predetermined number of nodes (e.g., 10 nodes), and an output layer configured for binary classification (e.g., 2 nodes for binary mapping).

During enrollment, each classifier in the bank is trained using the subject's biometric features as positive samples and synthesized impostor features as negative samples. However, using real impostor samples in their original form presents privacy risks. Thus, impostor data may be synthesized directly from representative centroids of their clustered embeddings rather than using their original biometric scans or features. More specifically, all available impostor feature vectors may be grouped into n clusters using K-means clustering with Euclidean distance, yielding a set of centroids of the embeddings {c₁, c₂, . . . , cₙ}. For example, n=300 clusters may be used based on the elbow method for clustering. These centroids capture the essential variation of the impostor domain while mitigating the storage of personally identifiable traits. Then, synthetic impostor samples may be generated by interpolating between each pair of centroids (cᵢ, cⱼ). Both linear and spherical interpolation methods may be employed to generate these synthetic samples, shown at equations (2) and (3), respectively, to provide a diverse set of impostor features for training while preserving privacy:

$${f}_{LERP} ( α ) = ( 1 - α ) {c}_{i} + α {c}_{j} \qquad ( 2 )$$

$${f}_{SLERP} ( α ) = \frac{sin ( ( 1 - α ) θ )}{sin ( θ )} {c}_{i} + \frac{sin ( α \cdot θ )}{sin ( θ )} {c}_{j} \qquad ( 3 )$$

The training process for each bit classifier involves assigning a random target bit value (0 or 1) and training the corresponding neural network to output that value when presented with the subject's biometric features (e.g., at operations 1006 and 1008 described above). The training may utilize standard optimization techniques (stochastic optimization) with appropriate learning rates (e.g., 0.001) and weight decay parameters (e.g., 0.0001) to prevent overfitting. To enhance the stability and reliability of the generated keys, data augmentation techniques may be applied during training. These may include geometric transformations such as rotation, scaling, translation, and elastic deformation, as well as photometric adjustments including brightness and contrast variations applied to the incoming vascular images. Such augmentations help the classifiers generalize better to natural variations in biometric presentation.

To reliably reconstruct keys during authentication, the same feature extraction pipeline described above processes the query biometric sample to generate a feature vector. This vector is then passed through the trained classifier bank, with each classifier producing a binary output corresponding to its assigned bit position. The concatenation of these binary outputs forms the reconstructed biometric key.

The stability of this approach may be enhanced through the use of error correction mechanisms or consensus techniques when multiple classifiers produce conflicting outputs. In some implementations, the system may require exact bit-wise matches for authentication, while in others, a predetermined Hamming distance threshold may be employed to accommodate minor variations.

When multiple biometric modalities are available (e.g., both DOT measurements and high-resolution vascular imagery, as enabled via the unitary scanning system 200), the key generation process may incorporate fusion techniques to combine information from different sources. Early fusion may combine raw sensor data before feature extraction, intermediate fusion may merge features from different modalities, and late fusion may combine the outputs of separate classifier banks trained on different modalities. The resulting biometric keys generated through this process exhibit high entropy and retain stability, making them suitable for cryptographic applications while maintaining the privacy-preserving properties of the overall system.

The template-free biometric verification methods described herein may be adapted for use with various biometric scanning technologies beyond vascular biometrics (e.g., fingerprint, iris, face, etc.); the foundations of the vector space operations, lattice generation, and closest vector point calculations remain applicable regardless of the underlying biometric capture mechanism, provided that the biometric data can be converted into a stable bit/vector representation through appropriate feature extraction algorithms.

The scalability of this approach extends to both single-modal and multi-modal biometric systems, wherein different types of biometric sensors may be combined to enhance security and accuracy. In some aspects, biometric scanners that operate on different physical principles may be incorporated, such as combining optical, thermal, electrical, and/or acoustic sensing methods. The deep learning architectures described herein may additionally be adapted to process various types of biometric data by adjusting the neural network structures, training datasets, and feature extraction pipelines to accommodate the specific characteristics of different biometric modalities. This enables the template-free verification protocol to be deployed across diverse applications and hardware platforms while maintaining the fundamental security properties of the protection scheme described above.

While the systems and methods above have been described and disclosed in certain terms and have disclosed certain embodiments or modifications, persons skilled in the art who have acquainted themselves with the disclosure, will appreciate that it is not necessarily limited by such terms, nor to the specific embodiments and modification disclosed herein. Thus, a wide variety of alternatives, suggested by the teachings herein, can be practiced without departing from the spirit of the disclosure, and rights to such alternatives are particularly reserved and considered within the scope of the disclosure.

When introducing elements or embodiments, the articles “a,” “an,” “the,” and “said” are intended to mean that there are one or more of the elements. The terms “comprising,” “including,” and “having” are intended to be inclusive and mean that there may be additional elements other than the listed elements.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively, or in addition, a component may be implemented by several components.

The above description illustrates embodiments by way of example and not by way of limitation. This description enables one skilled in the art to make and use aspects of the disclosure, and describes several embodiments, adaptations, variations, alternatives and uses of the aspects of the disclosure, including what is presently believed to be the best mode. Additionally, it is to be understood that the aspects of the disclosure are not limited in its application to the details of construction and the arrangement of components set forth in the following description or illustrated in the drawings. The aspects of the disclosure are capable of other embodiments and of being practiced or carried out in various ways. Also, it will be understood that the phraseology and terminology used herein is for the purpose of description and should not be regarded as limiting.

It will be apparent that modifications and variations are possible without departing from the scope defined in the appended claims. As various changes could be made in the above constructions and methods without departing from the scope of the present disclosure, it is intended that all matter contained in the above description and shown in the accompanying drawings shall be interpreted as illustrative and not in a limiting sense.

In view of the above, it will be seen that several advantages are achieved and other advantageous results attained.

The Abstract and Summary are provided to help the reader quickly ascertain the nature of the technical disclosure. They are submitted with the understanding that they will not be used to interpret or limit the scope or meaning of the claims. The Summary is provided to introduce a selection of concepts in simplified form that are further described in the Detailed Description. The Summary is not intended to identify key features or essential features of the claimed subject matter, nor is it intended to be used as an aid in determining the claimed subject matter.

What is claimed is:

1. A biometric scanning system for generating an image of a user's vasculature, the biometric scanning system, comprising:
a strap configured to be secured around an extremity of the user, the strap comprising a plurality of near-infrared light emitting diode (LED) banks;
an imaging unit comprising a near-infrared camera, the imaging unit configured to be releasably connected to the strap and positioned against the user's skin;
a biometric scan processor; and
a memory, wherein the memory is a non-transitory computer-readable storage medium communicatively coupled to the processor and storing software including instructions which, when executed by the processor, enable the system to perform functions comprising:
selectively activating the LED banks; and
selectively activating the near-infrared camera to capture images of the user's vasculature as illuminated by the LED banks.

2. The biometric scanning system of claim 1, wherein the plurality of LED banks comprises a dorsal LED bank, at least two lateral LED banks, and a ventral LED bank.

3. The biometric scanning system of claim 2, wherein the extremity is a wrist, wherein the dorsal LED bank is configured illuminate the user's vasculature from a back side of the wrist, wherein the lateral LED bank is configured to illuminate the user's vasculature from sides of the wrist, and wherein the ventral LED bank is configured to illuminate the user's vasculature around the near-infrared camera.

4. The biometric scanning system of claim 3, wherein the functions further comprise:
sequentially activating each LED bank in tandem with the near-infrared camera to capture a plurality of images, the plurality of images comprising:
a dorsal image stack comprising a plurality of dorsal images captured during illumination of the dorsal LED bank, the plurality of dorsal images including at least two images taken at different exposures;
a lateral image stack comprising a plurality of lateral images captured during illumination of the at least two lateral LED banks, the plurality of lateral images including at least two images taken at different exposures;
a ventral image stack comprising a plurality of ventral images captured during illumination of the ventral LED bank, the plurality of ventral images including at least two images taken at different exposures; and
generating a dorsal high-dynamic range (HDR) image from the dorsal image stack, generating a lateral HDR image from the lateral image stack, and generating a ventral HDR image from the ventral image stack.

5. The biometric scanning system of claim 4, wherein the processor is configured to generate the HDR images using perceptual exposure fusion.

6. The biometric scanning system of claim 4, wherein the functions further comprise generating a pseudo-RGB image.

7. The biometric scanning system of claim 6, wherein generating the pseudo-RGB image comprises:
assigning the dorsal HDR image, lateral HDR image, and ventral HDR image to a color channel selected from a group consisting of a red channel, a green channel, and a blue channel; and
combining the dorsal HDR image, lateral HDR image, and ventral HDR image to form the pseudo-RGB image;
wherein each of the HDR images are assigned to different color channels.

8. The biometric scanning system of claim 1, further comprising a user command module, the user command module comprising:
a command processor;
a first actuator configured to initiate an enrollment process when activated;
a second actuator configured to initiate a verification process when activated; and
a display communicatively coupled to the command processor and configured to provide operational feedback and status messages during biometric scanning processes.

9. A unitary biometric scanning system for generating an image of a user's internal anatomy, the unitary biometric scanning system comprising:
an imaging unit including a housing and a near-infrared camera located within the housing;
a plurality of imaging clusters, each imaging cluster comprising at least one near-infrared light emitting diode (LED) and at least one near-infrared sensor;
a processor and memory, wherein the memory is a non-transitory computer-readable storage medium communicatively coupled to the processor and storing software including instructions which, when executed by the processor, enable the biometric scanning device to perform functions comprising:
selectively activating the imaging clusters to perform diffuse optical tomography measurements of tissue structures within the user; and
selectively activating the near-infrared camera to capture images of vascular patterns within the user.

10. The unitary biometric scanning system of claim 9, wherein each imaging cluster comprises four near-infrared LEDs arranged in a square configuration and one near-infrared sensor positioned centrally within the four near-infrared LEDs.

11. The unitary biometric scanning system of claim 10, further comprising a strap connected to the housing, the strap comprising a plurality of imaging clusters distributed along an inner face thereof, wherein the system is a wrist-worn system configured to image the internal anatomy of the user's wrist, and wherein the plurality of imaging clusters are directed inwardly with respect to the person's wrist when the device is worn.

12. The unitary biometric scanning system of claim 10, wherein the strap comprises first and second strap portions extending outwardly from the housing on opposing sides thereof, the first and second strap portions configured to wrap around the user's wrist and releasably connect to secure the system thereto.

13. The unitary biometric scanning system of claim 10, wherein the near-infrared camera is configured to receive and detect light emitted from the near-infrared LEDs after it has passed through tissue or blood within the user.

14. The unitary biometric scanning system of claim 10, further comprising a near-field communication module positioned within the housing and configured to transmit biometrically-derived cryptographic keys to nearby access control systems for proximity-based authentication.

15. The unitary biometric scanning system of claim 10, wherein the imaging unit further comprises a ventral LED bank positioned on an inner face of the housing.

16. The unitary biometric scanning system of claim 15, wherein the housing defines an imaging port configured to permit the near-infrared camera to peer outwardly from within the housing, and wherein the ventral LED bank comprises an annular array of near-infrared LEDs surrounding the imaging port.

17. A method of granting or denying access to a safeguarded entity, the method comprising:
training a machine learning model using a plurality of vasculature images to generate, upon receiving an image of a person's vasculature, a unique and stable bit string associated with the person, wherein successive images of the person's vasculature will, when taken as input into the machine learning model, result in corresponding successive outputs of the same stable bit string;
scanning, using a biometric scanning system, the person's vasculature to yield an image thereof, the scanning system comprising a biometric scan processor and a memory coupled thereto, the memory comprising the machine learning model;
generating, using the machine learning model at the biometric scanning system, the stable bit string associated with the person;
receiving, by an access control system configured for guarding the safeguarded entity, the stable bit string associated with the person;
searching, by the access control system, a key list to determine if the stable bit string is present therein; and
granting, by the access control system, the person access to the safeguarded entity if the stable bit string is present in the key list.

18. The method of claim 17, wherein the machine learning model comprises a convolutional neural network, and wherein generating the stable bit string comprises extracting features from the image of the person's vasculature using the convolutional neural network.

19. The method of claim 18, wherein training the machine learning model includes training a bank of binary classifiers for replacing the stored template, each binary classifier corresponding to a specific bit position in the stable bit string.

20. The method of claim 18, further comprising encrypting and transmitting a hash of the stable bit string from the biometric scanning system to the access control system to enable the ensuing cryptographic functions.
